(19) United States
(12) Patent Application Publication
(10) Pub. No.: US 2020/0100822 A1
(43) Pub. Date: Apr. 2, 2020

(54) FASTENERS AND SYSTEM FOR PROVIDING FASTENERS IN BONE

(71) Applicant: Kenneth I. Lipow, Fairfield, CT (US)

(72) Inventor: Kenneth I. Lipow, Fairfield, CT (US)

(21) Appl. No.: 16/591,614

(22) Filed: Oct. 2, 2019

Related U.S. Application Data
(60) Provisional application No. 62/740,059, filed on Oct. 2, 2018.

(51) Int. Cl. A61B 17/86; A61B 17/70; A61B 34/32

(52) U.S. Cl. CPC A61B 17/864; A61B 17/7005; A61B 2017/681; A61B 34/32; A61B 17/8605

(57) ABSTRACT

Fastening devices for bone reconstruction that include a head portion; a centrally located rod having a length, a first end and a second end, the rod extending from the head portion at the first end of the rod; a plurality of threads extending around the centrally located rod; and a plurality of angled fenestrae residing within one or more of the plurality of threads to provide an angled fenestrated fastener for bone reconstruction. Computerized surgical systems, methods and equipment for fully-robotically implanting the angled fenestrated fasteners of the invention into a predetermined (i.e., premapped) bone with complete precision.

The present invention claims priority from U.S. Provisional Patent Application Ser. No. 62/740,059, the contents of which are incorporated herein in their entirety by reference thereto.

### BACKGROUND OF THE INVENTION

### 1. Field of the Invention

The present invention relates to bone and joint repair, and in particular, to fasteners, computerized mapping and navigational systems and equipment for implanting fasteners into a bone or joint.

### 2. Description of Related Art

The spine is a series of vertebrae extending from the skull to the small of the back. The spine encloses the spinal cord and provides support for the thorax and abdomen. There are four main regions of the spine which include the cervical spine, thoracic spine, lumbar spine, and sacral region.

The cervical spine relates to the region of the neck. The thoracic spine includes 12 vertebrae that provide stability and structural support to the upper back, as well as being attached to the rib cage to protect vital organs. The lumbar spine is at the lower back providing spinal motion and support for the torso. It is divided between five motion segments (i.e., L1-L5), with the lower segments taking the most wear and stresses. Attached to the lumbar spine is the sacral region. The sacral region includes the sacrum bone which connects the spine to the lower half of the body. Sacroiliac joints connect the sacrum bone to the two halves of the pelvis to form a three-dimensional structure.

Vertebrae residing within the different spinal regions are vastly different from each other. Due to these vast differences, the anatomy and physiology of vertebrae must be determined prior to surgical repair, along with determining the vertebral bone strength. There are several factors impacting bone strength including, for instance, macro-structural properties (e.g., mass, size and mineral (Ca+) density), micro-structural properties (e.g., cancellous bone structure and properties, cortical structure and properties), as well as bone material quality or properties. These bone properties may include mineralization (e.g., crystal size, structure, and homogeneity), bone matrix properties (e.g., fiber type, fiber age, collagen cross-linking and non-collagen molecule concentration), and marrow cell properties (e.g., osteoblasts, osteoclasts, stem cells, etc.).

In determining the anatomy and physiology of vertebrae prior to surgery, factors to be determined may include bone type and bone structure. Vertebrae bodies include two types of bone, namely, cortical bone and cancellous bone (i.e., trabecular). Cortical bone forms the hard outer shall and is the strongest, densest form of the bone. Cancellous bone is internal tissue of the skeletal bone and is an open cell porous network. It is weaker and less dense than cortical bone. Trabeculae of the cancellous bone forms a three-dimensional latticework (i.e., cancellus) of voids or interstices inside the vertebrae.

In surgical repair of bone, consideration must be given to the trabeculae found in the vertebral pedicle. The pedicle is a bone that connects the lamina to two short bone portions extending from sides of the vertebral body and forming the vertebral arch. Pedicles also form the base of the articular processes, which are the points of articulation for the ribs. Care must be given in surgical repair of the pedicle as it resides close to the sensory, motor and reflex nerves along the spine. As such, optimal precision in performing surgical procedures on the spine is imperative.

Surgical procedures have long been practiced on bone. The pathology of bone lends itself to self-healing. Over the years various medical equipment and procedures have been developed to help bone mend faster and heal more reliably. For instance, procedures have included the use of autogenous bone, bone fusion, bone grafting procedures, homograft fusions, and the use of surgical nails, rods, pins, bolts, screws and the like. Often, bolts and screws are used in combination with medical grade nails or rods for fixation to various bones including, for instance, the humeral bone or the spine.

Typically, pedicle bolts and screws are used as adjuncts to spinal fusion surgery as a means of anchoring a spinal segment. These screws or bolts may be placed through the vertebrae pedicle alone or in combination with grafting. However, the use of screws in repairing or fixation into the vertebrae pedicle is often problematic due to variations and differences from one pedicle to the next. These differences may include differences in pedicle shapes, sizes, and pedicle anatomy that make it difficult to obtain proper screw fixation within the pedicle.

Other differences include variations in angles from one pedicle to the next may differ. This angle degree variation often makes it difficult to obtain proper screw fixation as the varying angles result in different stresses being applied to the pedicles. Irregularities in the pedicle internal architecture also adversely affect screw fixation, as well as these irregularities disturbing navigational trajectories, particularly within computerized procedures that require manual. Still another problem with use of bolts and screws in pedicle surgery is that often the bolts and screws do not sufficiently go into or fixate within the pedicle dense bone.

In surgically implant fasteners, computer systems and mechanical medical equipment are often implemented in the process of surgically implanting medical grade bolts and screws into bone. These computer systems and medical equipment are often used to help guide the surgeon on location of where the surgeon must insert the bolt or screw into bone. Currently available computer guided systems include, for instance, computer guided spinal fixation, computer assisted screw and/or tumor resection navigation, computer guided biopsy instrumentation, and the like.

However, computer guided surgical systems are limited as they require manual surgical execution. That is, the surgeon is required to manually perform the surgical procedure merely with the aid and guidance of the computer systems and medical equipment. This leads to the potential of human error, incorrect or incomplete screw insertions, extended surgical times and costs. The computer aided or guided surgical systems are also subject to human and operating room limitations, as the surgeon may need to react to events occurring during a surgical procedure or within the operating room that may increase occurrence of error.

Accordingly, a need exists in the art for improved medical equipment, medical procedures, and medical systems that provide accurate results, and decreased operation times and costs.

### BRIEF SUMMARY OF THE INVENTION

The disclosed embodiments of the invention relate to fastening devices for bone reconstruction that include a head portion; a centrally located rod having a length, a first end and a second end, the rod extending from the head portion at the first end of the rod; a plurality of threads extending around the centrally located rod; and a plurality of angled fenestrae residing within one or more of the plurality of threads to provide an angled fenestrated fastener for bone reconstruction.

In other embodiments of the invention relates to computerized surgical systems, methods and equipment for fully-robotically implanting the angled fenestrated fasteners of the invention into a predetermined (i.e., premapped) bone with complete precision.

### BRIEF DESCRIPTION OF THE DRAWINGS

The features of the invention believed to be novel and the elements characteristic of the invention are set forth with particularity in the appended claims. The figures are for illustration purposes only and are not drawn to scale. The invention itself, however, both as to organization and method of operation, may best be understood by reference to the detailed description which follows taken in conjunction with the accompanying drawings in which:

FIG. 1 illustrates a medical grade bolt of the prior art.

FIGS. 2A and 2B illustrate prior art medical grade screws having conventional passive fenestrae in the rod portions thereof.

FIGS. 3A and 3B illustrate additional prior art medical grade screws having conventional passive fenestrae within the rods.

FIGS. 4A and 4B illustrate medical grade screws of the invention having active angled fenestrae only within the threads of such screws in accordance with the invention.

FIGS. 5A and 5B illustrate alternate medical grade screws of the invention having active angled fenestrae within threads and the rod in accordance with the invention.

FIG. 6 illustrates a medical grade bolt having active angled partially fenestrated openings in accordance with embodiments of the invention.

FIG. 7 illustrates fastener threads having active angled fully fenestrated openings in accordance with other embodiments of the invention.

FIGS. 8A and 8B illustrate the sharp edges of the angled fenestrae within a fastener for actively cutting and scooping bone material during fastener implantation in accordance with the invention.

FIG. 9 illustrates a fastener in accordance with one or more embodiments having both passive fenestrae and the active angled fenestrae of the invention.

FIG. 10 depicts graphed implant performance of various angled fenestrated fasteners of the invention.

FIG. 11 is a flow chart depicting the present robotic navigation and surgical implantation procedures in accordance with embodiments of the invention.

FIG. 12 illustrates radiographic imaged posterior vertebrae for confirming targeted bone/joint segments to be operated on during the mapping and planning phases of the robotic pedicle implant procedures in accordance with the invention.

FIG. 13 illustrates a matching unique key probe for use in the present robotic navigation and surgical implantation systems and methods of the various embodiments of the invention.

FIG. 14 illustrates a top down view of an operating room having the present computerized robotic navigation and surgical implantation systems and devices in accordance with embodiments of the invention.

### DESCRIPTION OF THE PREFERRED EMBODIMENT(S)

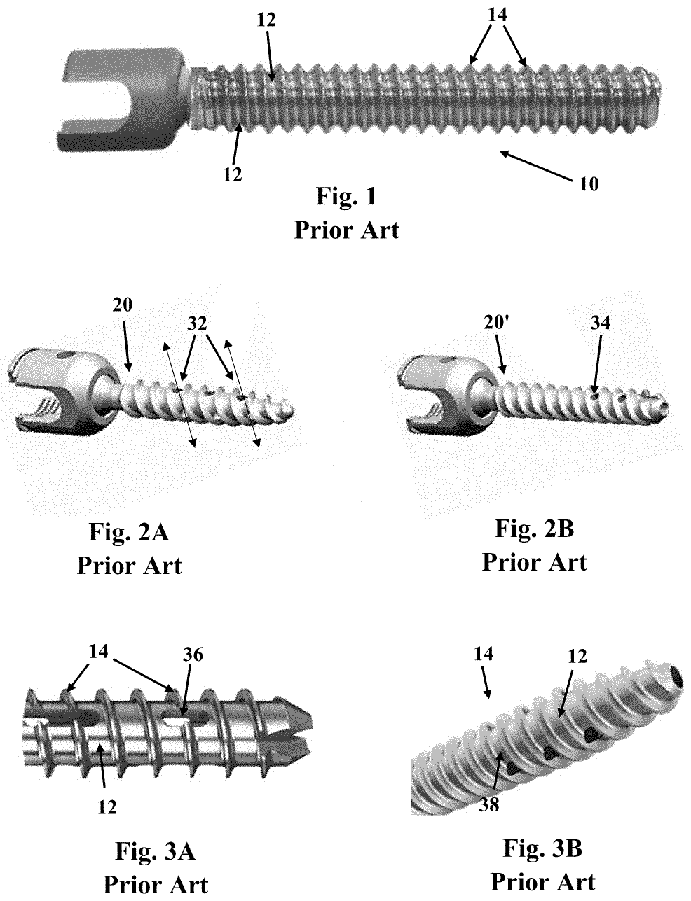

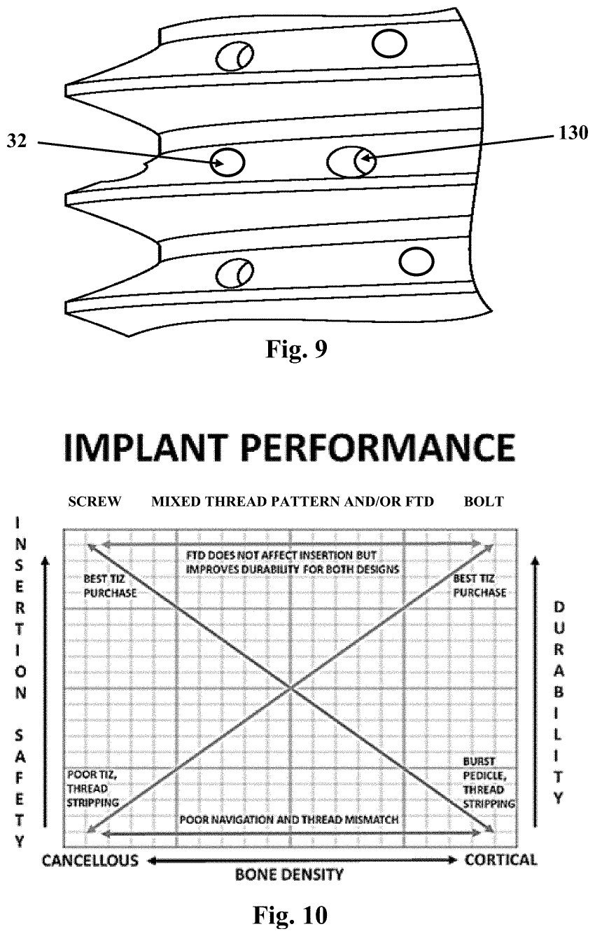

In describing the preferred embodiment of the present invention, reference will be made herein to FIGS. 1-14 of the attached drawings, as well as the drawings detailed herein below.

The various embodiments of the invention overcome problems of known computer systems, medical equipment and surgical hardware, particularly in connection with surgical procedures involving dense bone, such as, pedicle dense bone. In one or more embodiments the invention is directed to improved medical equipment, medical procedures, and medical systems that provide accurate results, and decreased operation times and costs.

In one or more embodiments the invention is directed to improved fenestrated fasteners, and in particular, improved fenestrated bolts and screws. Referring to FIGS. 1A to 3B, medical grade bolts and screws having passive fenestrations (i.e., openings) have been used over the years in surgical procedures. The fenestrations may be provided in a bolt 10 or they may be provided in screws 20, 20′, 22, 24 as shown in FIGS. 2A to 3B. The bolts 10 and screws 20, 22, 24 include a number of threads 14 residing and rotating around a centrally located shaft or rod 12 portion (hereinafter referred to as “rod 12”) of the bolt/screw. Further as shown, the rod 12 portion of each bolt and screw has a constant diameter from the head to the tip portions thereof.

Referring to the drawings, the screw 20 of FIG. 2A includes fenestrations 32 that extend fully through the rod 12 portion of screw 20, while FIG. 2B shows screw 20′ having fenestrations 34 extending partially through the rod 12. FIG. 3A depicts a screw 22 of the prior art having fenestrations 36 that reside both through the rod 12 portion of the screw and entirely through selected threads 14 of such screw. Still further, FIG. 3B shows screw 24 having fenestrations 38 that reside partly within both the rod 12 and the threads 14. That is, known bolts and screws have fenestrae (singular: fenestra; plural: fenestrae) that reside entirely within the rod 12 of the bolt/screw, or at least partially within the rod 12 thereof.

It has now been found conventional bolts 10 and screws 20, 20′, 22, 24, as shown in FIGS. 1 to 3B, having all or at least a part of the passive fenestrae residing within the rod 12 thereof are subject to failure over time. It is believed that this is due to exerted stresses on the implanted bolt/screw in combination with a bolt/screw having part of its central rod 12 removed forming a weaker implant that is more susceptible to damage and/or breakage. It has further been found that known bolts/screws having fenestrae within the rod 12 portion may become fully embedded in bone, such as pedicle bone. These fully embedded bolts/screws are irreversibly fixated within the bone, and nearly impossible to remove without undesirably destroying the bone from which it is being removed.

In overcoming this problem, the invention is directed in one or more embodiments to fasteners having fenestrae formed only within the threads thereof. The fenestrae of the invention are formed at axial angles, such that, the fenestrae openings of the invention have at least one or more sharp edges for etching bone material as the fastener is being implanted. Each angled fenestra of the invention are active fenestra since they cut/etch bone material, and then scoop up such removed bone material into the angled openings. As such, the present active angled fenestrae 130 perform an active role in the implant procedure since they cut and provide more bone matter into the angled fenestrae 130, as compared to the conventional passive fenestrae described in relation to FIGS. 1-3B.

That is, the conventional passive fenestrae of known fasteners are formed, or drilled, straight into the fastener with respect to a surface area of the fastener rod 12. As such, the fenestrae 32 opening sidewalls meet the outer surface of the bolt/screw rod portion at passive interconnections that do not cut or scoop bone materials. Rather, these fenestrae 32 have opening surface areas that merely allow bone material to fall into the fenestrae. The amount of bone material falling into the conventional passive fenestrae 32 is much less as compared to the amount of bone material that is gathered and scooped into the present angled fenestrae 130 of the invention since the angled fenestrae 130 perform an active role in the implantation process. In the conventional passive fenestrae 32, it has been found that insufficient amounts of bone material may fall into the fenestra 32, which leads to longer healing times and increased incident of implant failure or even breakage.

In one or more embodiments, the invention is directed to medical fasteners having angled fenestrae (openings) 130 in one or more surface areas of the fastener. The angled fenestrae may be provided on the threads of the fastener, the rod of the fastener, or both the threads and rod thereof. The fastener may be a bolt or a screw, or any other device for fixation into bone matter. The fastener may have a bone growth coating on a surface area thereof, and may have a bone growth coating around or inside one or more of the angled fenestrae openings of the invention.

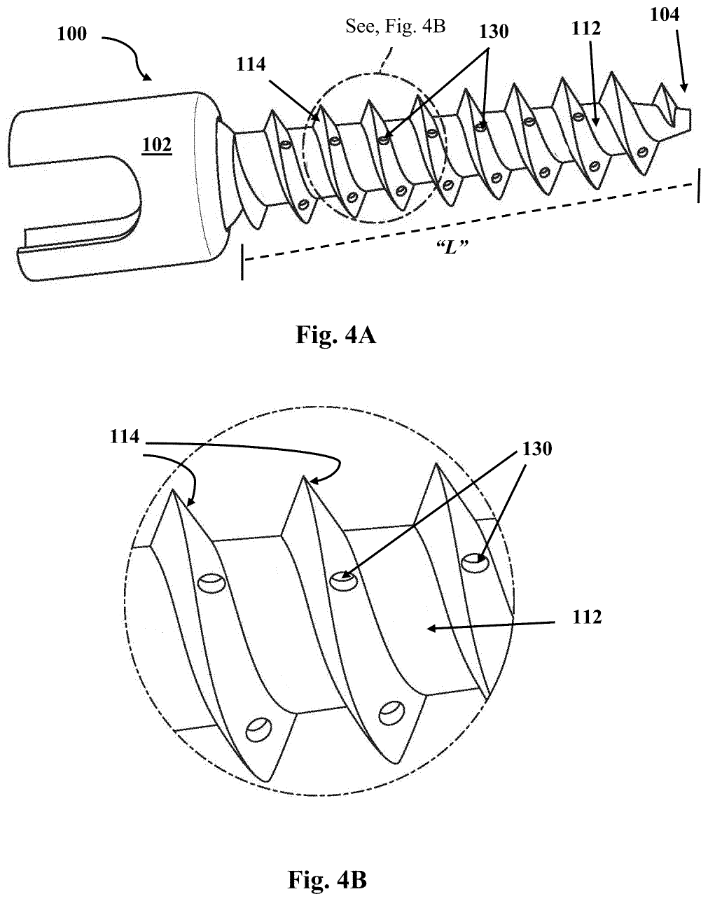

Referring now to FIG. 4A a fenestrated screw 100 in accordance with one or more embodiments is shown having the angled fenestrae 130 of the invention residing only within the threads 114 of such screw 100. As shown the rod 112 (i.e., core) of fenestrated screw 100 is free of any fenestra/openings, thereby maintaining rigidity and reliability of the screw 100 over time. The angled fenestrae are further shown in the exploded view of FIG. 4B. These fenestrae 130 reside within the threads 114 whereby the fenestrae angles are in a single transverse axial direction. It should be appreciated that some of the fenestrae 130 may be provided in the opposite direction in the event during implant the direction of insertion is reversed, thereby allowing bone matter to also be scooped within and deposited into the reverse directional fenestrae residing at an opposite angle to that of fenestrae 130.

Also as shown in FIG. 4A, the fenestrated screw 100 is provided with a rod 112 portion that has a conical configuration along its entire length “L”. That is the diameter of the rod 112 is larger at the head 102 portion of the screw 100 than it is at the tip 104 portion of such screw 100. This conical-shaped rod 112 enables a more secure and rigid fixation into the bone and/or joint.

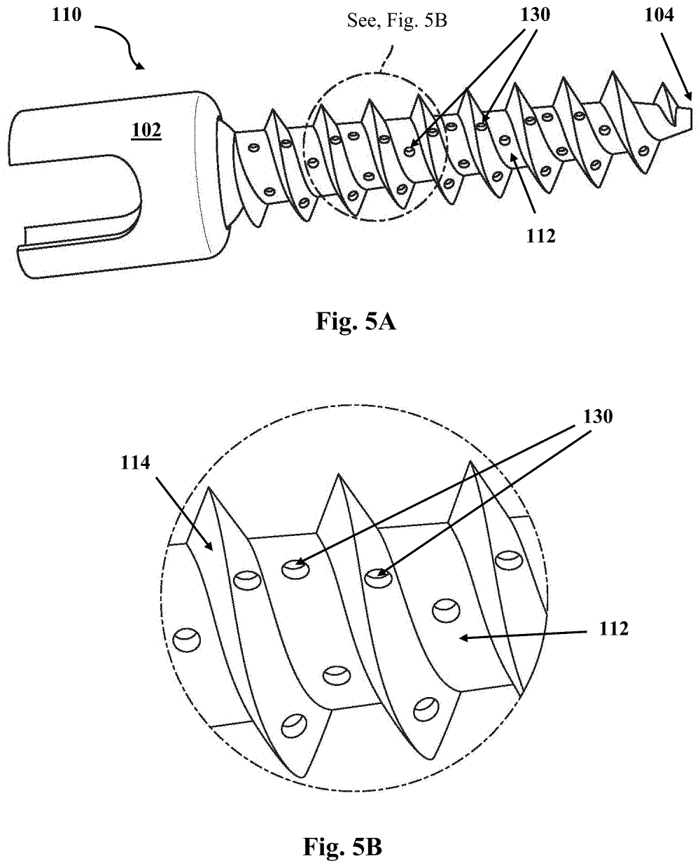

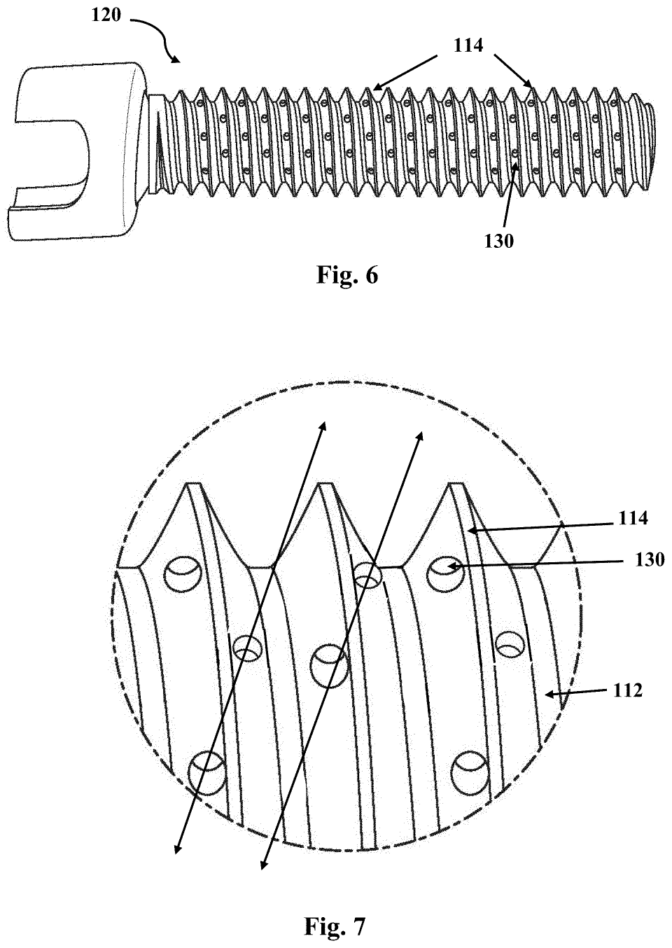

In one or more other embodiments, the present fenestrated screws 110 may include the active angled fenestrae 130 both within the threads 114 and the rod 112 of such screw 110, as shown in FIGS. 5A and 5B. Depending upon the type of bone the screw is to be inserted into, certain embodiments of the present angled fenestrae 130 may reside in both thread portions and rod portions. As shown in FIG. 6, the angled fenestrae 130 of the invention may also be provided within the threads 114 residing on a bolt 120 of the invention. In the various embodiment, the active angled fenestrae 130 may be provided only within the thread portions on the bolt, or alternatively they may be provided in the threads and rod portion (not shown) of the bolt.

Referring to FIG. 7, in embodiments of the invention the angled fenestrae 130 may be angled fully fenestrated openings that traverse through the entire thread 114. These fully fenestrated openings penetrate one side of a thread, extend through the thread, and exit such thread at an opposite thereof. The fully fenestrated openings 130 may be formed in a straight line through the thread, in an angled line, or any type of patterned opening through the thread. In other embodiments, the angled fenestrae 130 may be angled partially fenestrated openings that penetrate only partially into the thread 114, as shown at least in FIGS. 4A and 4B.

Whether the angled fenestrae 130 of the invention are provided as angled fully fenestrated openings, angled partially fenestrated openings, provided within the thread only, provided within both the thread and rod, provided within the rod only, or provided within a screw or bolt, or any combination thereof, each angled fenestra 130 has at least one angled sharp edge that cuts bone matter upon the angled fenestrated fasteners being implanted into bone. This cutting may be by etching, scraping, grating, and the like, whereby bone material is removed from the bone and is taken up and into the angled fenestrae 130 that performs active functions during the implantation process.

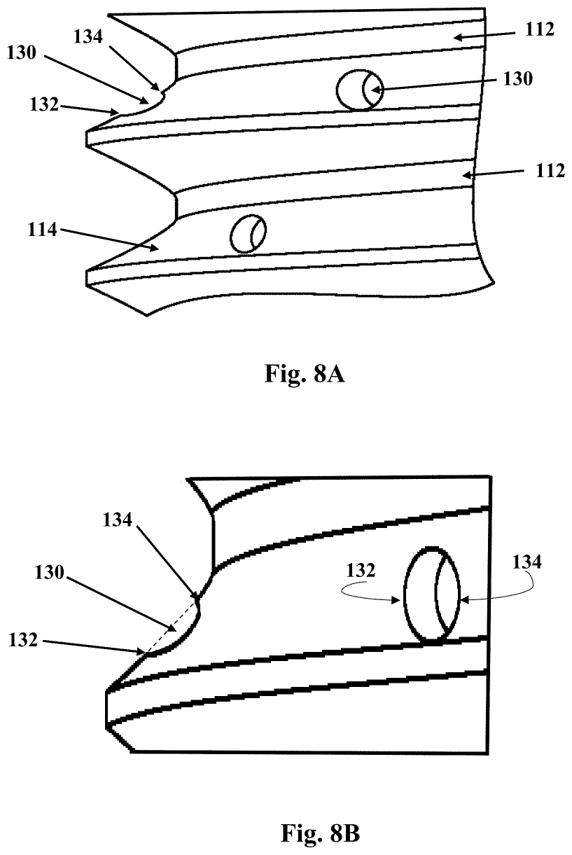

As shown in FIGS. 8A and 8B, in one or more embodiments the angled fenestrae 130 may have at least a first sharp edge 132 and a second sharp edge 134. Either together, or each sharp edge taken individually, provides the ability to cut and scoop living bone material during the surgical procedure of inserting/implanting the fastener (e.g., bolt or screw) into such bone. That is, the rotational forces of inserting the angled fenestrated fasteners into the bone presses the sharp edges 132, 134 against the bone resulting in bone material being cut and scooped into the angled fenestrated openings 130. In the various embodiments, these sharp edges 132, 134 may be sharp edge points, sharp edge regions, and/or sharp edge sections.

In still other embodiments, the angled fenestrae 130 of the invention may be used in combination with conventional fenestrae 32 to provide additional bone ingrowth regions and attributes to the implanted fastener over time. In one or more embodiments, the combination of fenestrations 130 of the invention and those of the prior art may be provided only within the threads of the fastener (e.g., threads of the bolt, or threads of the screw). By providing the angled fenestra 130 of the invention only in the threads, the surface area of the fastener (i.e., metal fastener material) is increased and applied forces during implantation are distributed over the entire core length as there are no fenestra therein.

In the various embodiments of the invention, the angled fenestrated fasteners of the invention may be provided with a variety of different shapes, sizes, diameters, lengths, and the like. That is, the angled fenestrated fasteners 130 (e.g., angled fenestrated bolts and screws) may be customized to a plurality of different angled Fenestrated Thread Designs (“FTD”) that meet criteria and parameters of the bone anatomy, densities, sizes, pathologies, and/or physiology into which such fastener is to be inserted/implanted. While not meant to be limiting, the lengths of the angled fenestrated fasteners, diameters of the angled fenestrated fasteners, number of threads and distances therebetween, shapes and sizes of the threads, and the like, may all be varied in one or more embodiments of the present angled fenestrated fasteners. Further, the threads 114 may have a single thread angle, thread pitch, and/or pitch crest, or alternatively the angle, pitch and/or crest may be variable along the length of the rod 112.

In one or more embodiments, as described in detail below, the invention is also directed to computerized surgical systems for fully-robotically implanting the angled fenestrated fasteners of the invention into a predetermined (i.e., premapped) bone with complete precision. These computerized surgical mapping and navigational systems may predetermine a size, location, and physiological parameters of a bone into which the axial angled fenestrated fasteners of the invention is to be implanted. The computerized surgical systems and equipment then fully-robotically perform the implantation procedure. That is, the computerized surgical systems and equipment are not computer-aided wherein the surgeon still needs to preform or assist in surgery. Rather, the computerized surgical systems and equipment of the invention are fully automated surgery performed using only robotics. In doing so, precise parameters of implantation are determined, and then the systems and equipment of the invention determine a size, angle, direction, and the like that the fastener is to take in precise implantation of the invention, as described in detail below.

FIG. 10 illustrates the graphed implant performance of the present angled fenestrated fasteners of the invention. The angled fenestrated screws 110 of the invention may be implanted by driving and splitting bone during implantation. During this process, as the screw 100 is rotated during insertion, the angled fenestrae 130 cut and scoop the living bone material into such angled fenestrated openings 130. In one or more embodiments the angled fenestrated screws 110 of the invention are suitable for use in soft cancellous bone. The graphed results of FIG. 10 show the safety and durability of the angled fenestrated bolts 120 into dense cortical bone, as well as the safety and durability of angled fenestrated screws 110 of the invention into soft cancellous bone. That is, the angled fenestrations 130 of the invention do not deleteriously affect the insertion of the fastener, but rather improve the durability of both the resultant implanted screws 110 and bolts 12 of the invention.

In embodiments that provide angled fenestrated bolts 120, a channel is bored into bone at the predefined location and angle, and then the channel is tapped at the root diameter in the dense bone subcortical and cortical zones to provide threading. In one or more embodiments the channel may be bored, then threaded, and the bolt implanted therein all by use of the fully automated computerized surgical mapping and navigational systems and equipment of the invention. The resultant tapped channel may be slightly smaller than a diameter of the bolt 120. For instance, the bolt diameter may be about 1 mm upsized (i.e., larger) than the tapped diameter of the channel. This enables the angled fenestrae 130 of bolt 120 to actively cut and scoop living bone during the process of insertion/implantation into the tapped (threaded) channel within the bone.

Once the bolt 120 with fenestrae 130 is fully inserted, the bone material deposited into the fenestrae and the surrounding bone tissue grow into the fenestrae 130 to firmly set and fixate the bolt 120 of the invention in position within the bone. In one or more embodiments, the present angled fenestrated bolts 120 of the invention are particularly suitable for use in dense vertebrae bone, such as implantation into pedicle dense bone. It has been found that implantation of the angled fenestrated bolts 120 are safer and improve dense bone implantation procedures and longevity. It has also been found that bolts 120 provide a more rigid and durable fixation within pedicle dense bone, as compared to conventional passive screws 20. In one or more embodiments, the bolts 120 of the invention may be longer than conventional screws 20, such that the bolts 120 extend deep into the pedicle bone. In comparison, conventional screws tend be too short within the pedicle bone and fit too loosely therein, all of which increase bone or joint reconstruct failure incident rates.

In the various embodiments of the invention, the active angled fenestrations 130 residing within the fastener of the invention increase the extent to which such fasteners are embedded into the bone/joint being repaired/reconstructed. This is due to the active angled fenestrae 130 being positioned, oriented, shaped and sharpened to cut and scoop living bone into the fenestrae opening, which in one or more embodiments resides in the threading plane when inserted. The bone material may partially fill such openings, or entirely fill such fenestrae openings, to increase the rate at which living bone grows inside the fenestrae.

The fasteners of the invention may be cannulated or non-cannulated, whereby bone ingrowth occurs faster and stronger within cannulated fasteners of the invention having the present angled fenestrae 130. Again, the fenestrae openings 130 may be provided with, or coated inside with, a bone growth enhancer to speed up the process of bone ingrowth inside the fasteners and fenestrae of the invention. The fenestrae openings 130 may also retain an epoxy therein for further adhering bone material to and securing the fastener into the bone/joint. The active angled fenestrations 130 improve the bioavailability of increased bone material inside the fenestrae and fasteners of the invention to provide a fastener that is a maturing biologic fastener that embed and locks into position within the bone/joint.

It has been found that the fasteners of the invention having the active angled fenestrae 130 improve mispercutaneous posterior screw performance and permanent construction rigidification. The fully embedded fasteners also facilitate screw/bolt and bone/joint reconstruction durability. The present fasteners may be used in patients with poor bone quality (e.g., osteoporosis, etc.) and are able to accommodate irregular shaped bones (e.g., an irregular shaped pedicle) since the present fasteners may be customizable to meet implantation and reconstruction criteria and parameters. The fasteners of the invention have been found to resist shearing, bending, torsional defects, alternating force fatigue, and loosening once fully embedded into the bone/joint construct or reconstruction.

In one or more other embodiments the invention is directed to fully automated computerized surgical mapping and navigational systems, equipment, and methods of use thereof. The present computerized robotic systems surgically implant medical fasteners into bone using robotics only, whereby the robotic surgery achieves precision, accuracy and better fixation of the implanted fastener, as compared to manual surgical procedures and/or computer-aided surgeries (i.e., those that require manual surgery in combination with computerized equipment). The present fully automated robotic surgical system of the invention improves surgical operating standards by eliminating human error or operating room errors. The robotic surgical system also provides increased surgical safety, enhances operating room conditions, increases surgery speed and precision, and is cost effective.

It has been found that the present fully robotic surgical computerized systems and methods are less invasive as compared to known approaches of surgically implanting fasteners, such as, entirely manual procedures, computer-aided or computer-guided procedures that still require manual labor, and/or mechanical procedures. Conventional manual procedures, or computer-aided or computer-guided manual procedures, are often disruptive due to human limitations and/or human error. For instance, manual procedures are often disruptive as the surgeon may need stop procedures for human evaluation, recalibrate equipment, operating disruptions may occur, the surgeon may become fatigued by overwork or use of heavy and cumbersome equipment, and the like. Human error may also result due to mistake or fatigue, as well as the surgeon being unable to perform and obtain precise surgical measures. For instance, in a manual procedure where the spatial resolution is at least 0.6 mm, a tap error of +/−2% results in an inaccuracy of up to 62% in implanting a 6.5 mm screw. All of this in the conventional approaches of implanting fasteners in bone or a joint may lead to incorrect or incomplete fastener insertions, and extended surgical times and costs.

In one or more embodiments the computerized robotic surgical mapping and navigational systems, equipment, and methods of the invention provide one or more of the following advantages over the above conventional implantation approaches. The fully robotic surgical systems of the invention provide consistent and repeatable results with increased accuracy over the prior art. The present systems are faster than manual (surgeon) procedures or those requiring manual implementation. The present systems utilize stabilized equipment such that the methods of performing surgery using the instant robotic surgical mapping and navigational prevents tremors, drifts, and/or wobbling that may occur in manual or manual assisted surgeries.

The robotic surgical systems, equipment, and methods of the invention also provide an uninterrupted, controlled and consistent force fastener insertion. In manual procedures pressures or force may undesirably fluctuate, potentially leading to an improperly implanted fastener. The present invention provides an instantaneous bone specific responsive force as the systems of the invention obtain, analyze and plan a mapped navigational route that takes into account varying bone parameters (e.g., different bone types, different densities, porosities, etc.)

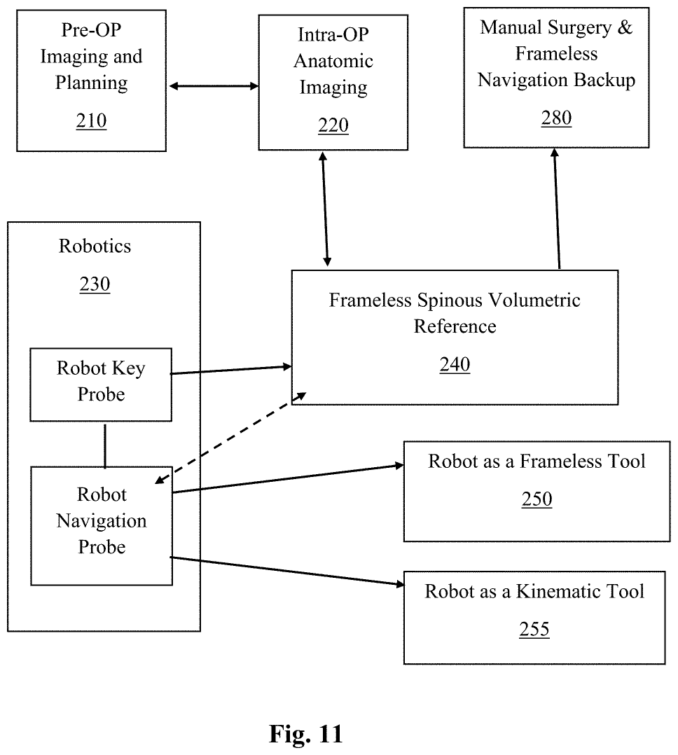

Referring to FIG. 11, a flow chart for the present robotic navigation and surgical implantation procedures in accordance with the invention is shown. While not meant to be limiting, the flow chart of FIG. 11 is described in relation to robotic pedicle implant procedures of the invention.

The first step in robotic navigation and surgical implantation of the invention is to determine the installation parameters for each fastener prior to surgery. In Step 210 the pre-operative phase occurs wherein preoperative imaging and planning is performed and calculated by the instant computerized mapping and navigational systems. In doing so, high quality or high-resolution images of the bone/joint to be repaired are obtained such as, for instance, by CT Scan, MRI, and the like. The imaged regions to be surgically repaired are analyzed for parameters including, but not limited to, bone type, shape, density, anatomy, physiology, porosity, etc. Using this information, the required fastener(s) types are determined (e.g. determine whether a screw is to be used, bolt, type of screw/bolt, shapes and sizes of screw/bolt, etc.).

Also, planned in this pre-operative phase is the trajectory start and target points for implanting the determined fastener. The optimal fit and engagement of the selected fastener is also determined and pre-planned prior to the instant robotic surgery. The computerized mapping and navigational systems of the invention are able to calculate and provide a precise, unique insertion formula for each fastener (bolt and/or screw) that is to be implanted in accordance with the planned pre-operative parameters. The calculated formulas may vary from implant to implant. These formulas take into account, and enable, optimal implant geometries and tulip positioning for the ideal reconstruction/construct build.

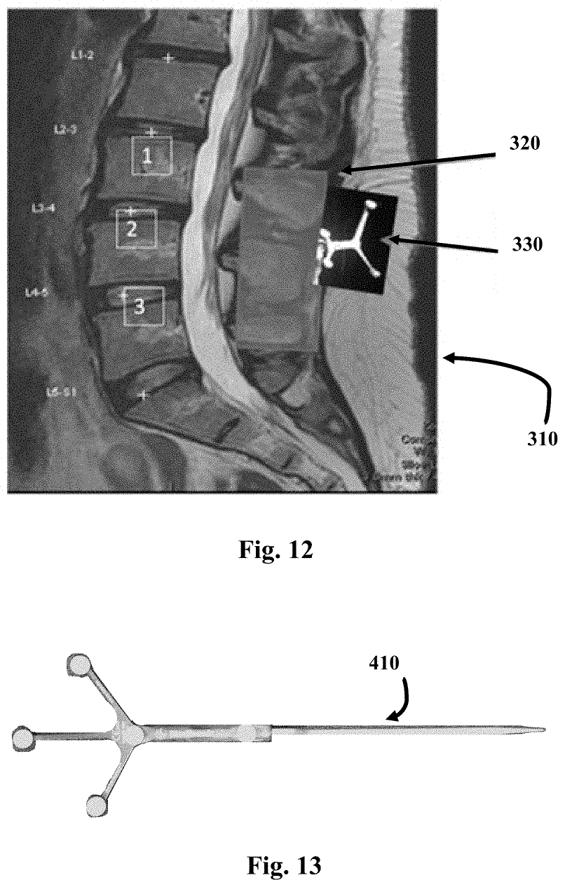

Step 220 includes the intra-operative phase where anatomic imaging occurs. As shown in FIG. 12, the posterior vertebrae is skeletonized, followed by radiographically 310 confirming the targeted segments to be operated on. Operative access is then gained to the target pedicles, followed by fixing a robotic clamp 320 on the spine with an integrated frameless volumetric reference 330. The intraoperative high-resolution scan is obtained with the spine registration. In one or more embodiments, the robotic clamp 320 is a 3-segmented robotic claim for mechanical spinal fixation. It provides rigidification to the targeted vertebrae to be operated on. The spinous volumetric reference 330 provides reference for the present frameless navigational scan and kinematics of the invention. Steps 210 and 220 may be compared to one another to detect and identify any changes in the vertebral alignment prior to proceeding to ensure robotic accuracy.

The robotic positioning device of the present may then be registered (i.e., linked) to the present computerized mapping and navigational system in Step 230. The robotic components of the invention include a robotic key probe and a robotic navigation probe. Referring to FIG. 13 a matching unique key probe 410 of the invention is depicted and has the identical navigation coding as the robotic navigation probe. The robotic positioning device of the invention is positioned over the operation table. This robotic positioning device may have robotically moving components, navigational guiding and a display unit. The robotic device is locked to the spinous clamp and the floor or table, and is then powered on. The present robotic surgical computerized systems automatically recognize the robotic device as a navigational tool. In one or more embodiments, one or more additional robotic devices may be added to the systems and methods of the invention, whereby each robotic device is recognized by their own unique encoding and fixation to the distal robotic arm.

In Step 240 the robotic device is registered to the frameless spinous volumetric reference 330. In doing so, both the unique robotic key probe 410 and the frameless robotic navigation probe are registered to the spinous volumetric reference prior to starting any surgical procedure. The robotic key probe 410 is an exact duplicate of the navigational reference on the robot. The robot device is automatically recognized as the navigational tool from the initial optical robotic key probe 410 registration, and is mechanically linked to the spinous clamp. The robotic key probe may transfer the submillimeter navigation functions to the internal kinematics of the spinous clamp to allow navigation of the patient's spinal anatomy. Using internal kinematics avoids any line of sight issues during operational procedures. In one or more embodiments the tool sequence may be the same for a procedure, or each tool may be uniquely encoded and recognized during an auto-exchange event. Further the use of forward and inverse kinematic navigation and motion control (i.e., the robotic device or robotic arm may move in various directions) improves the surgical field performance, efficiency and results.

The present robotic computerized mapping and navigational system then robotically bores, taps and implants/inserts the determined fastener into the identified bone/joint based on all of the pre-operational imaging, planning, and robotic device registrations. In the robotic operation and implantation of the fasteners of the invention, the present robotic computerized systems confirm all robotic start points and intended navigational trajectories. The fastener shaft sized kinematic navigation guided boring is activated at all target sites, and then tapping is performed. Each bored channel is provided with a 1 mm undersized kinematic navigation guided robotic system. Sensory displays are confirmed for safe boring and tapping. All chosen fasteners (screws/bolts) are inserted into adequate torque/force sensory display of the fastener engagement, and then the robot is removed from the surgical field. Imaging is performed to confirm precise implantation, and the reconstruction is finalized, neurosurgical decompressed, and the like.

The present robotic navigation system and computer system obtain various parameters for the desired or precise channel to be bored, and map out a plan or plans for each such channel. As such, each fastener and/or channel in the bone may be customized to fit the necessary requirements for repairing the bone. The robotic system implements the computer mapped plan and installs each of the fasteners. The systems of the invention map out the patient's anatomy and determine the configuration of each fastener needs, as well as the bore path and parameters for the specific bone(s) into which the fastener (e.g., a customized fastener) is to be implanted. The present invention may provide pedicle axial boring and optimal tapping with accurate cortical and subcortical fasteners of the invention to about 0.005 mm precision and accuracy. These results have been found to be repeatable. The robotic implantation system of the invention may further be used in combination with known instrumentation (e.g., an active ring form surgical effector) to provide the precise body part navigation implantation of the invention.

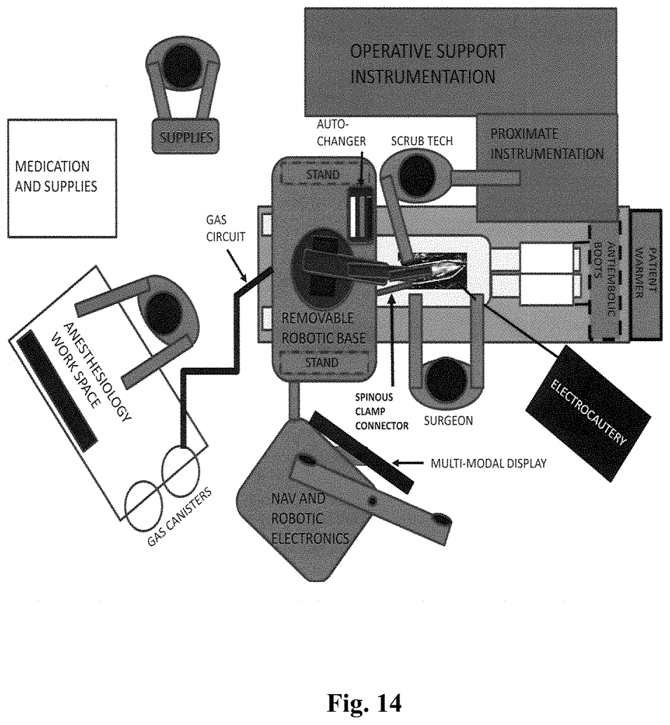

The present robotic navigation system and computer system provide real-time stress/strain measures during pedicle implant so that procedures can be adjusted and optimized for ideal and precise implantation. It provides indications of pedicle cortical breaching, monitors bone density for proper bot/screw fenestration selection, informs navigation from kinematic target positioning, provide instant final screw tightening confirmation and implant torque value matching, as well as graphed and visual results. Referring to FIG. 14, a top down view of a robotic navigation system and computer system is depicted in an operating room with medical personal. Placement of the removable robot base may be over the patients head and torso to yield the most effective coaxial symmetric motion patterns that are not disruptive to the operative process. In one or more embodiments, a suitable robotic device for use in the invention may be a MECA 500 Simulated Effector unit that includes a camera with a laser pointer, force/torque sensors, a tool driver shaft, and is suitable for use with the angled fenestrated fastener of the invention.

While the present invention has been particularly described, in conjunction with a specific preferred embodiment, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

Thus, having described the invention, what is claimed is:

1. A fastening device for bone reconstruction comprising:
a head portion;
a centrally located rod having a length, a first end and a second end, the rod extending from the head portion at the first end of the rod;
a plurality of threads extending around the centrally located rod; and
a plurality of angled fenestrae residing within one or more of the plurality of threads to provide an angled fenestrated fastener for bone reconstruction.

2. The device of claim 1 wherein the angled fenestrated fastener comprises an angled fenestrated screw.

3. The device of claim 1 wherein the angled fenestrated fastener comprises an angled fenestrated bolt.

4. The device of claim 1 wherein the plurality of angled fenestrae reside within a plurality of the threads.

5. The device of claim 1 wherein the plurality of angled fenestrae comprise a plurality of angled fully fenestrated openings.

6. The device of claim 1 wherein the plurality of angled fenestrae comprise a plurality of angled partially fenestrated openings.

7. The device of claim 1 wherein the plurality of angled fenestrae comprise a mixture of both a plurality of angled fully fenestrated openings and a plurality of angled partially fenestrated openings.

8. The device of claim 1 further including one or more angled fenestrae residing within the centrally located rod.

9. The device of claim 1 wherein the plurality of angled fenestrae each have one or more sharp edges for cutting bone material and scooping the cut bone material into the angled fenestrae.

10. The device of claim 1 wherein the plurality of angled fenestrae are coated with a bone growth material.

11. The device of claim 1 wherein the angled fenestrated fastener comprises an angled fenestrated screw having the plurality of angled fenestrae residing only within the plurality of threads

12. The device of claim 1 wherein the angled fenestrated fastener comprises an angled fenestrated screw having the plurality of angled fenestrae residing only within the threads of the screw.

13. The device of claim 12 further including one or more angled fenestrae residing within the centrally located rod

14. The device of claim 12 wherein the plurality of angled fenestrae each have one or more sharp edges for cutting bone material and scooping the cut bone material into the angled fenestrae.

15. The device of claim 1 wherein the angled fenestrated fastener comprises an angled fenestrated bolt having the plurality of angled fenestrae residing only within the threads of the bolt.

16. The device of claim 15 further including one or more angled fenestrae residing within the centrally located rod.

17. The device of claim 15 wherein the plurality of angled fenestrae each have one or more sharp edges for cutting bone material and scooping the cut bone material into the angled fenestrae.

18. The device of claim 1 wherein the angled fenestrated fastener is implanted into the bone by a computerized fully robotic implantation system.

19. A method for implanting a fastening device into bone comprising:
providing a fastener having a head portion, a rod portion, and a number of threads on the rod portion;
a plurality of angled fenestrae residing at least within the threads of the fastener;
digitally mapping out a precise location and implantation path on one or more bones using a robotic navigation system; and
robotically implanting the fastener into the mapped one or more bones to provide a fastener within the precise location and implantation path.

20. A system for implanting a fastening device into bone comprising:
a fastener having a head portion, a rod portion, and a number of threads on said rod portion;
a plurality of angled fenestrae residing at least within the threads of the fastener; and
a robotic navigation system that maps out a precise location and implantation path on one or more bones using a robotic navigation system.
